(19) United States
(12) Patent Application Publication
(10) Pub. No.: US 2026/0224348 A1
(43) Pub. Date: Aug. 6, 2026

(54) A Breast Implant Comprising a Triple Helix Spiral Structure

(71) Applicant: FASCIA INNOVATION SWEDEN AB, Sollentuna (SE)

(72) Inventor: Hans BOHLIN, Sollentuna (SE)

(73) Assignee: FASCIA INNOVATION SWEDEN AB, Sollentuna (SE)

(21) Appl. No.: 19/142,457

(22) PCT Filed: Dec. 15, 2023
(86) PCT No.: PCT/SE2023/000013

§ 371 Date: Jun. 23, 2025

(30) Foreign Application Priority Data
Dec. 23, 2022 (SE) 2230442-2

(51) Int. Cl. A61F 2/12

(52) U.S. Cl. CPC A61F 2/12; A61F 2210/00; A61F 2230/0091

(57) ABSTRACT

The invention relates to a human breast implant, and a method therefore, constructed with chambers, in a fascia triple helix structure, acting as through holes to body fluids. It solves the problem of allowing a flow of continuous interstitial fluid in and out of the implant.

### CROSS-REFERENCE TO RELATED APPLICATIONS

The instant application is a U.S. National Stage application of and claims priority to PCT/SE2023/000013, filed on Dec. 15, 2023, which is a PCT application of and claims priority to SE Application No. 2230442-2, filed on Dec. 23, 2022, the subject matter of both aforementioned applications is hereby incorporated by reference in their entireties.

### BACKGROUND OF THE INVENTION AND PRIOR ART

The present invention pertains to a human breast implant with multiple chambers to allow body fluids to diffuse into and out of it.

Current known human breast implants are of a solid construction often filled with a non-natural body fluid for instance made entirely of silicon or a silicon shell filled with a fluid salt/saline composition of for instance natrium chloride, whereby the implant and fluid may be accepted by a human body as body neutral. On a long-term basis, it is known that breast implants have leaked fluid and imploded causing severe damage/illness and discomfort to the bearer of such an implant.

Moreover, the solid shell of a breast implant may cause a buildup of scar tissue around its perimeter, with inflammations that later may give rise to dense collagenous tissue, fibrosis.

Prior art is defined through, for instance, the patent documents US 2006282164 A1, US2002091443 A1, US3189921 A, US 2017290650 A, US 2842775 A, and US3366975.

In US 2006282164 A1 to Seastrom, a breast implant is disclosed, in one embodiment providing a permeable shell filled with permeable micro beads. The breast implant lacks a firm structure as the beads can move inside its shell. Also, if the permeable shell is damaged or the beads are too small, they can release from the shell, and cause damage to living tissue. To overcome the problem of acting as a beanbag, which beans are due to gravity and other forces, it is partly supported with strands or channels. As they are partly supported, the beads can still deform a human breast implant inside the areas surrounded by strands and channels. It does not disclose fascia, a triple helix structure and that body fluids enter the implant in and out in a natural flow.

US 2002091443 A1 to Yoon, discloses a prosthetic implant for use in augmentation of the shape of a human body including an expandable body member having a first predetermined shape and a first volume in an expanded condition and a second volume in a second predetermined shape in a second volume smaller than the first volume. This implant has no firm structure, although permeable to body fluid there is no natural flow in and out of its shell for the fluids. Body fluids are only active to the swelling of the implant from its smaller volume to its expanded volume, i.e., they are not in a natural flow in and out of the implant.

US 3189921 A to Pangman describes preferably a breast implant where, in one embodiment, major parts of the implant are pervious or relatively pervious to blood. In another embodiment, it provides a material layer impervious to fluids and with substantially sealed pores. This patent document does not teach about fascia and that body fluids enter the implant in and out in a natural flow, i.e., merely to fill the implant with fluids to keep it in place in a human body.

Further, document US 2017290650 A describes an implant/prothesis to repair hernia ruptures with for instance elongated triple helix stripes with multiple tissue anchor points to build up strength around a rupture, which not suitable to spheric structures such as human breast implants. The document does not describe fascia and a natural flow of body fluids in and out of an implant through for instance diffusion.

Moreover, documents US 2842775 A, and US3366975 to Pragnan disclose a similar implant to the one cited in the latter. These documents disclose embodiments where an implant is impervious to body fluids, thus not teaching a natural flow of body fluids in and out of a breast implant.

### SUMMARY OF THE INVENTION

The present invention regards a human breast implant, which aims to lessen or remove the risk of upcoming discomforts/illnesses as mentioned.

Hence, set forth is a human breast implant constructed in the shape of a human breast. Hereby, provided is an implant with multiple chambers in a sponge-like structure, not soaking per se, to allow the interstitial fluid of a human body to diffuse/expand into and out of the sponge chambers in a more natural flow. Thus, reducing the buildup of dense collagenous tissue incapsulating the breast implant, by enabling the fluid to expand in a flow of fluid into and out of the breast implant in coherence with how the interstitial fluid of a human body operates in living tissue.

In one preferred embodiment of the present invention, the sponge-like biotensegrity structure is built up as a triple helix structure.

Furthermore, in one embodiment, a breast implant is provided, wherein the triple helix structure chambers and its outlining aligns to measurements coherent with the human fascia structure.

It also, in one embodiment, provides a breast implant, wherein the breast implant is constructed by a material that is known to be non-body repellent, utilized to manufacture human body implants/prothesis. Examples of those include micro poly urethan foams, silicon rubber and other known materials to a person skilled in the art.

Yet more, it provides in another embodiment that the structure of the breast implant is equipped/furnished with material conducting electricity for instance gold, silver, or copper in its entire shape or partially to mimic functions of the triple helix in the real fascia.

Further, it presents a method to the construction of a human breast implant in accordance with the above teaching.

### A BRIEF DESCRIPTION OF THE DRAWINGS

Henceforth, reference is had to the accompanying drawings throughout the present description for a better understanding of the present invention's embodiments, and given examples, wherein:

FIG. 1a schematically illustrates a breast implant in one view in accordance with an embodiment of the present invention; and

FIG. 1b schematically illustrates a breast implant in another view of the embodiment in FIG. 1a.

The following definitions utilized throughout the description are believed to have an established scientific meaning as expressed.

A scientific definition of an interstitium is conveyed through for instance being the fluid filled space between and around all cells that flows in and between all tissue and organs in an entire human/mammal body. It can be summarized as not providing empty spaces, but to being a confined continuous flow kept in place of a net structure of fiber proteins, mostly collagen in differing thicknesses and densities.

The following scientific articles are examples of them describing interstitium:
Evidence for continuity of interstitial spaces across tissue and organ boundaries in humans; Odise Cenaj, Douglas H. R. Allison, Rami Imam, Briana Zeck, Lilly M. Drohan, Luis Chiriboga, Jessica Llewellyn, Cheng Z. Liu, Young Nyun Park, Rebecca G. Wells & Neil D. Theise. The article is retrieved through the URL https://www.nature.com/articles/s42003-021-01962-0; and
Structure and Distribution of an Unrecognized Interstitium in Human Tissues;
Petros C. Benias, Rebecca G. Wells, Bridget Sackey-Aboagye, Heather Klavan, Jason Reidy, Darren Buonocore, Markus Miranda, Susan Kornacki, Michael Wayne, David L. Carr-Locke & Neil D. Theise. Scientific Reports volume 8, Article number: 4947(2018 ). The article is retrieved through the URL https://www.nature.com/articles/s41598-018-23062-6

The following scientific articles are examples of them defining tensegrity/biotensegrity:
Tensegrity I. Cell structure and hierarchical systems biology, Donald E. Ingber retrieved through URL https://journals.biologists.com/jcs/article/116/7/1157/27541/Tensegrity-I-Cell-structure-and-hierarchical;
A New Concept of Biotensegrity Incorporating Liquid Tissues: Blood and Lymph Bruno Bordoni, DPT, DO, PhD https://orcid.org/0000-0002-4949-5126bordonibruno@hotmail.com, Fabiola Marelli, DPT, DO, [ . . . ], and Roberto Castagna, DPT, DO;
https://journals.sagepub.com/doi/full/10.1177/2515690X18792838; and
Meaning of the Solid and Liquid Fascia to Reconsider the Model of Biotensegrity Bruno Bordoni, David Lintonbon, Bruno Morabito retrieved through https://assets.cureus.com/uploads/review_article/pdf/13292/1612427996-1612427993-20210204-18590-1of320f.pd.

Findings of structured water EZ water are derived to for instance Prof. Pollack and are taught in for instance URL https://www.pollacklab.org/research and discussed in Exclusion Zone Phenomena in water-A critical Review of Experimental Findings and Theories. To Elton et al URL https//www.ncbi.nlm.nih.gov.

In a collagen network and in cells, there exists a flow of water with. i.a., a variety of molecules. The water is per se structured water i.a. meaning that it is negative loaded, and thus contributing to electrical conductivity. Structured water is in symbiosis with i.a. proteins and exposed to infrared light (IR). An atom H+is split from H2O and the molecules structure as honeycomb like cake layers. Summarized its now H3O2-(H2O+OH—), and the loose H+ so to say makes up an anode pol. The structured water constitutes a crystalline gel like shape as a mixture, by Prof. Pollack called the fourth dimension of water. It exists everywhere in a body interstitial flow between fibers and cells. Also, inside of cells and cytoplasm. Therefore, the flow is an important part of a human body signal and communication system.

### DETAILED DESCRIPTION OF PREFERRED EMBODIMENTS

One study discusses that body fluids diffuse into and out of aperture especially in fascia, whereby one is provided by the paper “Structure and Distribution of an Unrecognized Interstitium in Human Tissues” to Petros C. Benias et al. This article was published at www.nature.com as article No. 4947, 2018 now referred to as Benias.

Benias describes in sum “the anatomy and histology of a previously unrecognized, though widespread, macroscopic, fluid filled space within and between tissues, a novel expansion and specification of the concept of the human interstitium.”

The triple helix structure is common in a human body for instance, the DNA is built on a so-called double helix, two nucleic acid strands that complement each other in a spiral. In a similar manner, the collagen molecule consists of three amino acid chains twisted/intertwined in a triple helix structure. Collagen is the most common protein creating the network surrounding and providing the structural framework for the cells and organs in a body. They provide the framework and structure to that permits movement and communication amongst cells of the body.

In Benias, it is taught that a human fascia system of the human body, amongst other organs and tissue, is subject to the findings of the expansion of the human interstitium, which is embraced by the present inventions breast implant.

It is appreciated that the present invention breast implant in one embodiment is constructed as a sponge-like item. Herein meaning that it is plastic/shaping, i.e., not soaking per se. As described in the below examples with reference to the attached drawings, the implant is built up in accordance with a fascia collagen triple helix structure.

Movability within the triple helix structure regarding collagen molecules and fibers are built in so called, “three-chains”, defined as three spirals twined/twisted in one direction. In a chain it is followed by three-chains twined in the other direction and so forth. This structure presents an ability to the structure to move, stretch and thereafter return to its original structure, like a twined/braided hemp rope, which structure the present invention mimics.

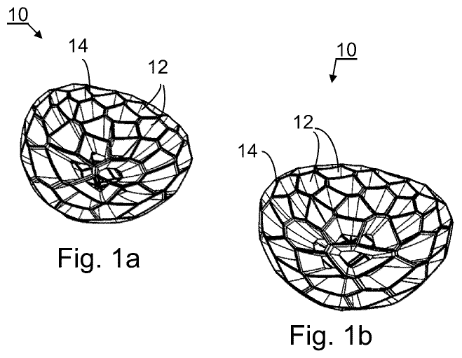

FIG. 1a schematically illustrates a breast implant 10 in one view in accordance with one embodiment of the present invention where multiple apertures/chambers 12 admit a free expansion in a natural flow of interstitial fluid into and out of the breast implant 10 around its perimeter and through it, which itself mimic functions of a collagenous triple helix 14 in the real fascia. A natural flow of interstitial fluid inhibits/decreases the possibility of an upbuild of dense collagenous tissue. A natural/constant flow of body fluids meant herein is a reference to how the human interstitium operates. Also, a natural flow in this context is provided by either one of for instance diffusion/blood pressure/osmosis and the exclusion-zone water (EZ) water phenomena.

Diffusion is a spontaneous process of transport/expansion of an element, herein body fluids, induced by random changes in the eigen movements of an element's atoms/molecules due to a molecule's energy content. Furthermore, the present invention natural flow also includes the biologic theory of EZ water properties and its electrical/signal assets as a factor in symbiosis with diffusion/blood pressure/osmosis as described above.

The fascia covers and permeates all organs and human/mammal body parts in a net/mesh/matrix like fashion, i.a., to uphold the structure and biotensegrity of organs and an entire body. Commonly, biotensegrity is defined by the words biological-tension-integrity, which determines a self-stabilization of the body. A principal for almost all living beings that demands tensegrity. A bearing structure built on tension and compression and therebetween a flow of interstitium. Biotensegrity provides what is considered as flexibility/movement in a triple helix structure in collaboration with interstitium in fascia.

Moreover, the fascia structure is built up of collagen fibers, which in turn consist of collagen molecules, formed like a triple helix 14 to promote conducting of current/electricity/energy/charges for instance as electrical pulses through its shape as a mesh or a braid in its basic form. Therefore, the present invention in one embodiment provides that the sponge like triple helix structure 14 material of the breast implant 10 is equipped/furnished with material configured for conducting electricity, for example, gold, silver, or copper in its entire shape or partially to mimic functions of the real fascia. Yet more, in one embodiment, the electric conductors should be thin so that the material of the breast implant 10 overcomes forces in the metal when the implant 10 is stressed/moved etc., not to be deformed. It is important in one embodiment of the present invention to provide a possibility to fully adhere to the EZ (i.e., Exclusion Zone) water theory by introducing metal strands built into the implant spirals to be able to convey electric signals in accordance with how fascia acts.

Hence, the breast implant 10 of the present invention in one embodiment combines the natural softness of a sponge in nature with the biotensegrity structure to keep the implant in its shape and not to implode, but to be elastic/plastic. One example shape of a breast implant is in the form of a teardrop, but others known in the art are also possible.

By mimicking the fascia, the present invention in one embodiment provides that the interstitium flow operates in the breast implant 10, which makes it more like living tissue in a body, and the implant 10 is more likely to be regarded as a natural part of the body than to be artificial, thus lessening or preventing the implant from encapsulation of malicious tissue such as an upbuild of dense collagenous tissue.

Summarizing, the finding behind the present invention in one embodiment of the invention makes it possible to manufacture a breast implant which through bio-tensegrity and a conductive structure upholds a flow of fluid and energy.

FIG. 1b schematically illustrates a breast implant 10 in a different view of the embodiment in FIG. 1a to illustrate that the interstitium operates on the implant 10 from all directions of its perimeter and through it in all possible directions in a spheric aspect.

It is appreciated that the triple helix structure 14 in FIGS. 1a and 1b is regarded as an exemplifying embodiment out of other possible ones within the attached set of claims. For example, other embodiments of the present invention can include all known possible variations of the triple helix structure 14 in layers to build up the breast implant 10. Seen in the attached drawings is a breast implant 10 showing a triple helix structure/chain 14, spiral like in its peripheral asymptotic area seen from above with four contact points between each three spirals when connected mimicking a fascia net. Triple helix spirals are consecutively connected through four contact/anchor points. Spaces between four anchor points and consecutive ones in layers make up chambers in accordance with the present invention. The triple helix spirals are twined/braid like a braided hemp rope in layers to build a breast implant body.

The braid may be tied with differential/variance, for instance loosely or closer tied in different areas of the implant in one embodiment, or evenly tied depending on how plastic/elastic the breast implant should be in different areas of it. It should present chambers between and amongst braids and braid layers. Those chambers are in connection with each other through the entire breast implant to permit the natural flow of body fluids in accordance with the present invention. Chambers may be of like sizes/measurements/dimensions or different ones throughout the implant.

To grasp the entire breast implant 10 as a triple helix structure 14, layers of spirals interconnected should be interpreted as being present in FIGS. 1a and 1b for instance as three-chain layers.

In one elaborated version of the triple helix structure 14 of the present invention as described with reference to FIGS. 1a and 1b, the breast implant 10 is constructed by multiple connected triple helix 14 braid layers in a density allowing the breast implant to be elastic and not too firm as taught in the latter.

The implant 10 could in one embodiment be of variating density, for instance denser in its core. Other shape variations of density can also be possible.

A manufacture of the breast implant of the present invention could be by 3D printing, molding, casting, foundry i.e., through methods known to a person skilled in the art for manufacturing of 3D items.

The dimensions of the breast implant 10 chambers in one embodiment regarding construction can be like in Mediterranean Sea sponges. In another embodiment of the present invention, they are coherent with for instance a mean value of a human fascia aperture dimensions or a chosen preferred dimension of them within nano meter (nm) to milimetter (mm) range.

Human fascia dimensions can for instance as an example be derived from ScienceDirect, Journal of Biomechanics, volume 77, 22 Aug. 2018, pages 69-75, “Site specificity of mechanical and structural properties of human fascia lata and their gender differences: A cadaveric study.” to Otsuka et al.

What is claimed is:

1-7. (canceled)

8. A human breast implant constructed in the shape of a human breast, the human breast implant comprising multiple chambers in a sponge-like biotensegrity structure configured to allow interstitial fluid in a human body to diffuse in a natural flow into and out of said sponge-like chambers, wherein said sponge-like biotensegrity structure is a triple helix spiral structure in layers interconnected in a fascia configuration, thereby decreasing the buildup of dense collagenous tissue incapsulating said breast implant by the fluid being configured to diffuse in said natural flow of fluid into and out of said breast implant in coherence with how interstitium operates in a human body.

9. A breast implant according to claim 8, wherein said triple helix structure chambers and its outlining mimics to measurements coherent with the human fascia structure, said measurements being within the values of a mean value of a human fascia aperture dimension or a chosen preferred dimension of them within a nanometer (nm) to millimeter (mm) range.

10. A breast implant according to claim 8, wherein the triple helix structure of the breast implant is equipped/furnished with material configured for conducting electricity in its entire shape or partially to mimic functions of the real fascia to transport signals from EZ-water properties in said natural flow.

11. A breast implant according to claim 10, wherein the material for conducting electricity is at least one of gold, silver, and copper.

12. A method to a human breast implant characterized by the step of:
constructing and manufacturing it in the shape of a human breast and furnishing it with multiple chambers in a sponge-like biotensegrity structure to allow human body fluids to diffuse in a continuous natural flow into and out of said sponge like chambers; and
wherein said sponge-like biotensegrity structure is constructed as a triple helix spiral structure in layers interconnected like a fascia through interstitial fluid of a human body, hereby decreasing the buildup of collagenous tissue incapsulating said breast implant.

13. A method according to claim 12, wherein said sponge-like bio-tensegrity structure is a triple helix spiral structure, having four contact points between each spiral, in layers interconnected like a fascia.

14. A method according to claim 12, wherein said sponge-like biotensegrity structure chambers and its outlining mimics to measurements coherent with the human fascia structure said measurements being within the values of a mean value of a human fascia aperture dimension or a chosen preferred dimension of them within nanometer (nm) to millimeter (mm).

15. A method in accordance with claim 12, wherein the triple helix structure of the breast implant is equipped/furnished with material configured for conducting electricity in its entire shape or partially to mimic functions of the real fascia to transport signals from EZ water properties in said natural flow.

16. The method in accordance with claim 15. wherein the material for conducting electricity is at least one of gold, silver, and copper.
